(19) United States
(12) Patent Application Publication
(10) Pub. No.: US 2013/0100409 A1
(43) Pub. Date: Apr. 25, 2013

(54) Method and System for Combining OCT and Ray Tracing to Create an Optical Model for Achieving a Predictive Outcome

(71) Applicants: Robert Edward Grant, Laguna Beach, CA (US); David Haydn Mordaunt, Los Gatos, CA (US)

(72) Inventors: Robert Edward Grant, Laguna Beach, CA (US); David Haydn Mordaunt, Los Gatos, CA (US)

(21) Appl. No.: 13/405,122

(22) Filed: Feb. 24, 2012

Related U.S. Application Data
(60) Provisional application No. 61/549,642, filed on Oct. 20, 2011.

(51) Int. Cl. A61B 3/10; G01B 9/02

(52) U.S. Cl. 351/221; 356/496

(57) ABSTRACT

A system and method are provided for combining the imaging capabilities of an Optical Coherence Tomography (OCT) device with the calculated results of ray tracing techniques. The combination is then used to derive a predictive refractive outcome for an optical model. The resultant optical model includes diopter power and size information for use in preoperative planning (e.g. a capsulotomy) and/or for the manufacture of an Intraocular Lens (IOL).

This application claims the benefit of U.S. Provisional Patent Application Ser. No. 61/549,642, filed Oct. 20, 2011.

### FIELD OF THE INVENTION

The present invention pertains to a system and method for creating an optical model of a substantially transparent object. More particularly, the present invention pertains to combining the imaging capabilities of an Optical Coherence Tomography (OCT) device, together with the calculated results of ray tracing techniques, to predict refractive outcomes with an optical model of an object. The present invention is particularly, but not exclusively, useful as a system and method for creating an optical model, with diopter power and size information, that can be used for preoperative planning (e.g. a capsulotomy) and/or for the manufacture of an Intraocular Lens (IOL).

### BACKGROUND OF THE INVENTION

Various diagnostic and therapeutic techniques are well known for use in ophthalmic procedures. In the application of each of these techniques, the ultimate purpose is to first clearly and precisely define the eye (anatomically and optically), and to then use the diagnostic information that is obtained to improve the refractive properties of the eye for vision correction. With this in mind, of particular interest for the present invention are the technologies of Optical Coherence Tomography (OCT) and ray tracing.

As is well known, OCT is an optical signal acquisition and processing method that is based on coherence interferometry techniques. Essentially, in brief overview, an OCT device collects light that is reflected from a target (i.e. a sample or specimen). The device then compares this reflected light with light that, is reflected from a reference. It happens that most of the light that is incident on the target, and on the reference, will be scattered light, rather than reflected light. Consequently, only the light that is reflected (non-scattered) from the target and from the reference will be coherent. Based on this fact, an interferometer is used in an OCT device to strip the scattered (non-coherent light) from the reflected light. An important result here is that the reflected (coherent) light can be used to image the target.

Apart from OCT, ray tracing is a well known method for calculating the path of a beam of light (i.e. a ray of light). In particular, ray tracing relies on the basic assumption that a ray of light will travel in a medium along a straight path. And, it will travel on this straight path through a distance in the medium, until a local derivative of the medium at a point on the beam path causes the direction of the ray to change. At that point, a new direction for the light ray is calculated and the basic assumption is repeated. This is an iterative process that is continually repeated until a complete path for the light ray has been calculated.

As used for the present invention, the methodologies of OCT and ray tracing are complementary. Specifically, using the imaging techniques of OCT, regions of varying light propagation with different reflectivity and absorption characteristics can be identified inside a substantially transparent object. These material properties of an object, along with optical characteristics of the light itself, such as intensity, wavelength and polarization, can be used for ray tracing calculations. Importantly, these calculations all lend themselves to computer processing. In the event, a consequence of ray tracing is a better understanding of the refractive properties of the object that is being evaluated.

A surgical procedure of particular interest for the present invention is a capsulotomy operation that is used for the treatment of cataracts. More specifically, such a procedure typically involves the removal of the cataract lens from its capsule bag in an eye. The removed lens is then replaced by an Intraocular Lens (IOL). In this exchange, measureable changes in anatomical dimension of the capsule bag are to be expected. Also, the IOL itself may have dimensional differences from the crystalline lens that was removed. Moreover, the IOL will have a different index of refraction from that of the anatomical lens that has been replaced. In the event, all of these differences will introduce refractive changes into the optical characteristics of the eye. And, these changes need to be accounted for in order to restore an appropriate vision quality for the patient.

In light of the above, it is an object of the present invention to provide a system and method for creating an optical model of a substantially transparent object (e.g. the crystalline lens of an eye) that can be used to predict a refractive outcome caused by dimensional and material change in the transparent object. Another object of the present invention is to provide a system and method for creating an optical model of a crystalline lens of an eye for use in preoperative planning (e.g. a capsulotomy). Still another object of the present invention is to provide a system and method for creating an optical model of a crystalline lens of an eye for use in the manufacture of an Intraocular Lens (IOL). Yet another object of the present invention is to provide a system and method for combining OCT and ray tracing to create an optical model that is easy to use, is simple to implement and is comparatively cost effective.

### SUMMARY OF THE INVENTION

In accordance with the present invention, a system is provided to create an optical model for analyzing and evaluating the propagation of light rays through a substantially transparent object. Included in the system are an Optical Coherence Tomography (OCT) device for creating an anatomical profile of the object, and a ray tracer for creating a refraction profile of the object. A computer/controller is also included for coordinating the operations of both the OCT device and the ray tracer. In particular, the computer/controller is employed to use the anatomical profile for calculating the refraction profile, and to then superpose the refraction profile onto the anatomical profile to thereby create the optical model of the object. As envisioned for the present invention, the resultant optical model can be modified by computer input to analyze and evaluate diopter power and size information that can be used in preoperative planning (e.g. a capsulotomy) and/or for the manufacture of an Intraocular Lens (IOL).

Structural components for the system of the present invention include an imaging unit that is used for scanning an imaging beam along a predetermined path through the transparent object. This is done to create an anatomical profile of the object. In detail, the anatomical profile will provide spatial dimensions of the object, and it will identify the location of structures which introduce refractive changes within the object. Preferably, the imaging unit is an Optical Coherence Tomography (OCT) device of a type well known in the pertinent art.

Along with the imaging unit, the system includes a ray tracer. Specifically, for purposes of the present invention the ray tracer calculates the beam paths for a plurality of rays as they would be affected by the anatomical profile. For this calculation, each ray comprises a contiguous sequence of ray segments. Importantly, each ray segment will have both a direction and a length. In this case, the direction of each ray segment is based on the uniquely identifiable material derivative (i.e. refractive changes) that is characteristic of the material at the ray segment's start point. Typically, the length of the ray segment will be arbitrarily chosen, and it can be less than approximately 100 microns.

As envisioned for the present invention, the process of ray tracing will be accomplished by following a computer program, and it will require the individual calculation of many ray segments. In general, as implied above, in addition to the consideration of physical properties of the object itself such as light propagation, reflectivity, and absorption characteristics, the calculation of a ray segment includes considerations of the light beam's characteristics such as intensity, wavelength, and polarization. The consequence of these considerations is that at its respective origin, each ray segment will likely have its own, respectively unique material derivative. This will certainly be the case where the refractive indexes of materials are abruptly different at their interface.

With the above in mind, a refractive profile is created in the following manner. First, a start point is selected having a predetermined location in the object. Next, a ray segment is advanced from the start point along a straight line through the object. As indicated above, the ray segment is advanced in a direction that is based on the material derivative of the object at the start point of the ray segment. The calculated ray segment then extends through a distance (e.g. 100 microns) from its start point to an end point. After the ray segment has been advanced, the resultant end point is then designated as a start point for a subsequent ray segment. Another iteration of calculations is then performed and a subsequent ray segment is advanced. This process is repeated until there is a contiguous sequence of ray segments that is sufficient to establish and identify a light ray. A plurality of such light rays is then considered together as a refraction profile for the object.

The entire process for creating an optical model is essentially computer controlled. In this process the computer employs ray tracing techniques to generate a refraction profile that is based on an anatomical profile which, in turn, is previously obtained using OCT techniques. The computer then combines the anatomical profile with the refraction profile, to thereby establish an optical model of the object. Once an anatomical profile (obtained by OCT) and a refraction profile (obtained by ray tracing) have been combined to create an optical model, the model can be used preoperatively for planning purposes or for designing an IOL.

In an operation of the present invention, dimensions in the anatomical profile of the optical model can be arbitrarily changed by computer inputs. In response, the computer employs ray tracing techniques to accordingly realign the refraction profile. Thus, any number of changes in the anatomical profile can be analyzed by additional ray tracing iterations as are necessary.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a schematic presentation of the functional aspects of the system and its method for creating an optical model in accordance with the present invention;

FIG. 2 is a representative anatomical profile as obtained by an OCT device for of a portion of an eye;

FIG. 3 is a ray-tracing exemplar for use as a portion of a refraction profile in accordance with the present invention;

FIG. 4A is representative of a pre-modified optical model obtained by superposing a simplified refraction profile onto a specified anatomical profile;

and

FIG. 4B is a representative optical model with an IOL modification for comparison with the optical model of FIG. 4A.

### DESCRIPTION OF THE PREFERRED EMBODIMENTS

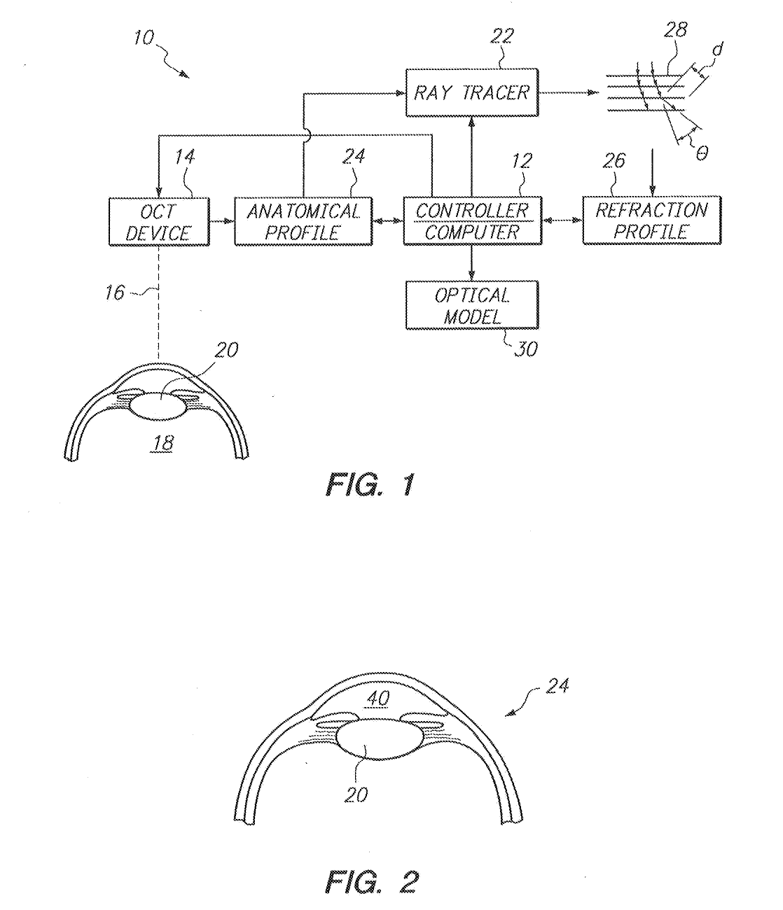

Referring initially to the FIG. 1, a system for combining Optical Coherence Tomography (OCT) with ray tracing techniques in order to achieve a predictive outcome is shown, and is generally designated 10. As shown, the system 10 includes a computer (controller) 12 that coordinates directly with an OCT device 14. For purposes of the present invention, it will be appreciated by the skilled artisan that the OCT device 14 can be any type of imaging device known in the pertinent art that is capable of generating three dimensional images of a substantially transparent object. In FIG. 1, the OCT device 14 is shown directing an imaging beam 16 toward an eye 18. More specifically, the imaging beam 16 is being directed toward the crystalline lens 20 of the eye 18. FIG. 1 also shows that the computer 12 is connected directly to a ray tracer 22.

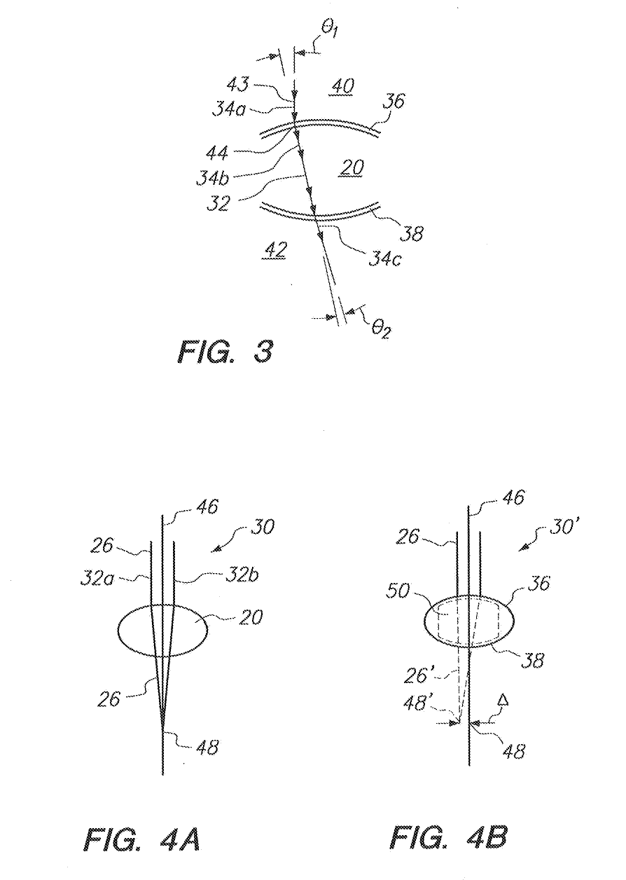

In an operation of the system 10, the computer 12 is first used to activate and control the OCT device 14. The purpose here is for the OCT device 14 and the computer 12 to interact with each other for the generation of an anatomical profile 24. In this case, this anatomical profile 24 will pertain to a substantially transparent object(s), such as the eye 18 and its lens 20, and will contain information pertinent to the lens 20 (object). In particular, the anatomical profile 24 is created to provide dimensions and measurements of the eye 18 and its lens 20 (object). Additionally, the anatomical profile 24 will also identify the locations, of various structures within the eye 18 and the lens 20 that introduce refractive changes to light, as the light passes through the lens 20 (object). Once the anatomical profile 24 has been created, the computer 12 then activates and controls the ray tracer 22 to generate and create a refraction profile 26. An exemplar 28 for the operation of the ray tracer 22, during a creation of the refraction profile 26, is shown in FIG. 1 and is discussed in greater detail below with reference to FIG. 3. After the refraction profile 26 has been created, it is superposed by the computer/controller 12 onto the anatomical profile 24 to establish the optical model 30.

In FIG. 2, a typical anatomical model 24 of an eye 18 is shown. Preferably, as intended for the present invention, the anatomical profile 24 will be generated by the OCT device 14. In any event, the optical model 30 will define different structures within the eye 18 (e.g. lens 20), and it will provide size and distance measurements regarding these structures. Though only the anterior portion of eye 18 has been shown in FIG. 2, it is to be appreciated that an optical model 30 can be generated for the entire eye 18, or for another portion of the eye 18.

FIG. 3 is a more detailed exemplar 28 which, for purposes of disclosure, shows a single light ray 32. As shown, the light ray 32 comprises a plurality of different ray segments 34, of which the ray segments 34a, 34b and 34c are exemplary. In FIG. 3, the light ray 32 is shown passing through the anterior capsule 36 of lens 20, through the lens 20, and through the posterior capsule 38. In this context, when considering the light ray 32, it is important to appreciate that each of the ray segments 34 has a direction and a length. More specifically, the direction of each ray segment 34 will be determined by a derivative (i.e. refractive index) of the material through which it is passing, and it will include a consideration of the optical characteristics of the light ray 32 (e.g. intensity, wavelength and polarization). On the other hand, the length of each particular ray segment 34 is arbitrary, and this length can vary from one ray segment 34 to another, as desired. For purposes of the present invention, it is to be appreciated that the length of a ray segment 34 may be less than about one hundred microns.

With the above in mind, consider the ray segments 34a and 34b in FIG. 3 as they pass from the anterior chamber 40 and into the crystalline lens 20. As is well known, aqueous in the anterior chamber 40, and the lens 20, have different indexes of refraction. Consequently, the direction of ray segment 34b will differ from that of the ray segment 34a. In FIG. 3, this difference is indicated by the angle θ₁. If, as assumed here, there is no substantial change in the refractive index of material along the path of light ray 32 as it passes through the lens 20, there will be no direction changes for ray segments 34 in the lens 20. As the light ray 32 exits the lens 20, however, the index of refraction of the vitreous 42, which is different from that of the lens 20, will change the direction of the ray segment 34c. This change is indicated by the angle θ₂.

As will be appreciated by the skilled artisan, when calculated by the ray tracer 22, the direction of each ray segment 34 is determined at its start point: For example, the direction of ray segment 34a will be determined based on the derivative that is calculated at its start point 43. The direction of ray segment 34b will then be determined based on the derivative that is calculated at its start point 44. This process then continues for a sequence of contiguous ray segments 34 until the light ray 32 is sufficiently defined. Note: for purposes of this disclosure, refractive changes that may have been caused by the anterior capsule 36 or the posterior capsule 38 have been assumed to be negligible. In actual practice, however, a consideration of these refractive contributions may become important as more precision is required.

Following the methodology generally outlined above, the paths for many different light rays (e.g. light ray 32) are similarly determined. In line with the above disclosure, the plurality of individual light rays that is determined by ray tracing techniques are then grouped together into the refraction profile 26. Specifically, this grouping is accomplished according to the respective dimensional and spatial relationships that are established by the anatomical profile 24. The optical model 30 is then created by combining the refraction profile 26 with the anatomical profile 24.

For an operation of the present invention, an optical model 30 of an eye 18 is first created as disclosed above. In FIG. 4A, a simplified optical model 30 is shown oriented on a reference axis 46. For this simplified optical model 30, the anatomical profile 24 is represented by the crystalline lens 20, and the refraction profile 26 is shown as light rays 32a and 32b passing through the lens 20. As shown in FIG. 4A, the refraction profile 26 of optical model 30 establishes a focal point 48 on the reference axis 46. As this point, for purposes of disclosure, the model 30 is ready for operational use.

In FIG. 4B, a modified optical model 30′ is shown (also oriented on the reference axis 46). In FIG. 4B, however, the model 30 (FIG. 4A) has been modified by using input from computer/controller 12 to simulate a capsulotomy wherein an Intraocular Lens (IOL) 50 is implanted between the anterior capsule 36 and the posterior capsule 38 of the eye 18. The computer/controller 12 then uses this computer simulation input to change the anatomical profile 24 as indicated by the short dash lines in FIG. 4B. The ray tracer 22 then recalculates a refraction profile 26′ (indicated by the long dash lines in FIG. 4B) that is based on the changed anatomical profile 24. The consequence of this is a modified optical model 30′ (FIG. 4B). In this example, the computer/controller 12 will then be able to determine any deviations that may have occurred, such as the deviation “Δ” which is shown as a movement of point 48 to point 48′.

In accordance with an operation of the system 10, computer simulations can be performed to predict and evaluate deviations “Δ” that may occur when material is removed from the eye 18, or when foreign material (e.g. IOL 50) is introduced into the eye 18. Thus, the system 10 can be used to predict the refractive effect of material and structural changes in the eye 18 and evaluate such changes for any of several purposes. In particular, using the system 10, an effective IOL 50 can be designed to accommodate actual refractive changes in the eye 18 that may be introduced during ophthalmic surgery. Also, the system 10 can be used to preplan this surgery. While the particular Method and System for Combining OCT and Ray

Tracing to Create an Optical Model for Achieving a Predictive Outcome as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A method for creating an optical model for the propagation of light rays through a substantially transparent object which comprises the steps of:
scanning an imaging beam along a predetermined path through the object to obtain information about the spatial dimensions and the location of structures within the object;
using the information obtained in the scanning step to create an anatomical profile for the object;
selecting a start point, wherein the start point has a predetermined location in the object;
advancing a ray along a straight line segment in the object from the start point to an end point, wherein the line segment has a direction determined by a material derivative of the object at the start point and the line segment has a predetermined length;
designating the end point as a start point after the advancing step;
repeating the advancing step and the designating step to create a refraction profile for the object; and
combining the refraction profile with the anatomical profile to establish the optical model of the object.

2. A method as recited in claim 1 further comprising the steps of:
changing the anatomical profile in the optical model;
recreating the refraction profile in the model based on the changing step; and
evaluating any refractive change indicated by the optical model in response to the changing step and the recreating step.

3. A method as recited in claim 1 wherein the material derivative is an index of refraction of the structure at the start point.

4. A method as recited in claim 1 further comprising the step of altering the derivative at the start point to account for properties of the ray wherein each property is selected from a group comprising intensity, wavelength, and polarization.

5. A method as recited in claim 1 wherein the predetermined length of the straight line segment is less than 100 microns.

6. A method as recited in claim 1 wherein the scanning step is accomplished by an Optical Coherence Tomography (OCT) device.

7. A method as recited in claim 6 wherein the selecting step, the advancing step, the designating step and the repeating step are accomplished by a ray tracer.

8. A method as recited in claim 7 wherein the OCT device and the ray tracer are controlled by a computer, wherein the combining step is accomplished by the computer, and further wherein the method is accomplished in accordance with a computer program run by the computer.

9. A method as recited in claim 1 wherein the object is a crystalline lens of an eye.

10. A method as recited in claim 1 wherein the optical model is used for the manufacture of an Intraocular Lens (IOL).

11. A system for creating an optical model for the propagation of light rays through a substantially transparent object which comprises:
an Optical Coherence Tomography (OCT) device for creating an anatomical profile for the object;
a ray tracer for creating a refraction profile for the object; and
a computer/controller for combining the anatomical profile and the refraction profile to create the optical model of the object.

12. A system as recited in claim 11 wherein control of the OCT device is accomplished by the computer/controller in accordance with a computer program comprising program sections for respectively scanning an imaging beam along a predetermined path through the object to obtain information about the spatial dimensions and the location of structures within the object, and using the information obtained in the scanning step to create the anatomical profile for the object.

13. A system as recited in claim 11 wherein control of the ray tracer is accomplished by the computer/controller in accordance with a computer program comprising program sections for respectively selecting a start point having a predetermined location in the object, advancing a ray along a straight line segment in the object from the start point to an end point, designating the end point as a start point after advancing the ray, and repeating the advancing of the ray and the designating of the start point to create the refraction profile for the object, wherein the line segment has a direction from each start point determined by a material derivative of the object at the start point, and the line segment has a predetermined length.

14. A system as recited in claim 11 wherein the computer/controller includes a computer program having program sections for combining the anatomical profile with the refraction profile to establish the optical model of the object; changing the anatomical profile in the optical model; recreating the refraction profile in the model; and evaluating any refractive change indicated by the optical model.

15. A system as recited in claim 11 wherein the object is a crystalline lens of an eye and the optical model is used for the manufacture of an Intraocular Lens (IOL).

16. A system for creating an optical model for the propagation of light rays through a substantially transparent object which comprises:
an imaging unit for scanning an imaging beam along a predetermined path through the object to create an anatomical profile of the object, wherein the anatomical profile includes spatial dimensions of the object and the location of structures within the object;
a ray tracer connected to the imaging unit for using the anatomical profile to uniquely identify a plurality of material derivatives at a plurality of respective, individually selected points in the object, and for calculating a ray segment at each selected point based on the material derivative of the object at the selected point, to create a refraction profile for the object based on the resultant plurality of ray segments; and
a computer connected to the imaging unit and to the ray tracer for combining the anatomical profile with the refraction profile to establish an optical model of the object.

17. A system as recited in claim 16 wherein each ray segment has a direction and a length, and wherein the length is less than approximately 100 microns.

18. A system as recited in claim 16 wherein each material derivative of the object includes factors pertinent to the object and are selected from a group comprising light propagation, reflectivity, and absorption characteristics of the object.

19. A system as recited in claim 18 wherein the calculation of a ray segment includes consideration of light characteristics selected from a group comprising intensity, wavelength, and polarization.

20. A system as recited in claim 16 wherein the anatomical model is selectively changed and the refraction profile is correspondingly recreated for evaluating any refractive changes caused by changing the anatomical profile.
